(19) United States
(12) Patent Application Publication
(10) Pub. No.: US 2020/0191760 A1
(43) Pub. Date: Jun. 18, 2020

(54) OPERATIONAL FLUIDS GAS ANALYSIS

(71) Applicant: Nabors Drilling Technologies USA, Inc., Houston, TX (US)

(72) Inventor: Tollef WINSLOW, Houston, TX (US)

(21) Appl. No.: 16/593,751

(22) Filed: Oct. 4, 2019

Related U.S. Application Data
(60) Provisional application No. 62/780,034, filed on Dec. 14, 2018.

(51) Int. Cl. G01N 33/00; E21B 49/08; E21B 43/34; E21B 21/06

(52) U.S. Cl. CPC G01N 33/0016; E21B 21/067; E21B 43/34; E21B 49/086

(57) ABSTRACT

A system that can include a gas extractor to extract a gas at a first temperature from an operational fluid, with the extracted gas containing vapor when it exits the gas extractor into a flow passage and a gas analyzer that receives the gas at a second temperature from the flow passage, where a temperature difference between the first temperature and the second temperature is less than 30 degrees Celsius. A system that can include a gas extractor to extract a gas from an operational fluid and a gas analyzer that receives the gas from a passage in communication with the gas extractor and analyzes the gas, where the gas is not subjected to conditioning between the gas extractor and gas analyzer.

### CROSS-REFERENCE TO RELATED APPLICATION

This application claims priority under 35 U.S.C. § 119(e) to U.S. Provisional Patent Application No. 62/780,034 entitled “Drilling Fluids Gas Analysis,” by Tollef WINSLOW, filed Dec. 14, 2018, which is assigned to the current assignee hereof and incorporated herein by reference in its entirety.

### FIELD OF THE DISCLOSURE

The present disclosure relates to systems and methods associated with the analysis of gases within drilling fluids circulated from subterranean formations.

### RELATED ART

Drilling for oil and gas typically involves the use of a drill string urged into a subterranean formation to form a wellbore. As the drill string advances into the wellbore, formation materials such as cuttings, gasses, and fluids removed from the wellbore are circulated from the bottom of the wellbore to the surface. Analysis of the formation materials has become of interest to drillers in order to improve wellbore planning and operations. However, traditional analysis performed on the formation materials is not meeting demands.

The drilling industry continues to demand improvements in wellbore planning and formation analysis. In particular, the drilling industry continues to demand improvements in gas analysis, which can assist drill operators in better drill rig and wellbore performance.

### BRIEF DESCRIPTION OF THE DRAWINGS

The present disclosure may be better understood, and its numerous features and advantages made apparent to those skilled in the art by referencing the accompanying drawings.

FIG. 1 includes a schematic view of a drilling rig in accordance with an embodiment as the drilling rig is engaged in wellbore operations.

FIG. 2 includes an enlarged schematic view of a portion of the drilling rig in accordance with an embodiment.

### DETAILED DESCRIPTION

The following description in combination with the figures is provided to assist in understanding the teachings disclosed herein. The following discussion will focus on specific implementations and embodiments of the teachings. This focus is provided to assist in describing the teachings and should not be interpreted as a limitation on the scope or applicability of the teachings. However, other embodiments can be used based on the teachings as disclosed in this application.

The terms “comprises,” “comprising,” “includes,” “including,” “has,” “having” or any other variation thereof, are intended to cover a non-exclusive inclusion. For example, a method, article, or apparatus that comprises a list of features is not necessarily limited only to those features but may include other features not expressly listed or inherent to such method, article, or apparatus. Further, unless expressly stated to the contrary, “or” refers to an inclusive-or and not to an exclusive-or. For example, a condition A or B is satisfied by any one of the following: A is true (or present) and B is false (or not present), A is false (or not present) and B is true (or present), and both A and B are true (or present).

Also, the use of “a” or “an” is employed to describe elements and components described herein. This is done merely for convenience and to give a general sense of the scope of the invention. This description should be read to include one, at least one, or the singular as also including the plural, or vice versa, unless it is clear that it is meant otherwise. For example, when a single item is described herein, more than one item may be used in place of a single item. Similarly, where more than one item is described herein, a single item may be substituted for that more than one item.

As used herein, “generally equal,” “generally same,” and the like refer to deviations of no greater than 10%, or no greater than 8%, or no greater than 6%, or no greater than 4%, or no greater than 2% of a chosen value. For more than two values, the deviation can be measured with respect to a central value. For example, “generally equal” refer to two or more conditions that are no greater than 10% different in value. Demonstratively, angles offset from one another by 98% are generally perpendicular.

Unless otherwise defined, all technical and scientific terms used herein have the same meaning as commonly understood by one of ordinary skill in the art to which this invention belongs. The materials, methods, and examples are illustrative only and not intended to be limiting. To the extent not described herein, many details regarding specific materials and processing acts are conventional and may be found in textbooks and other sources within the drilling arts.

In accordance with an aspect described herein, a system for use in subterranean operations can generally include a gas extractor and a gas analyzer. The gas extractor can be configured to extract a gas at a first temperature from an operational fluid. The extracted gas can contain vapor, such as water vapor, oil vapor, another vapor, or combinations thereof, as it exits the gas extractor into a flow passage. The gas analyzer can receive the gas from the flow passage at a second temperature. The temperature difference between the first temperature and the second temperature can be less than 30 degrees Celsius. In an embodiment, the temperature difference between the first temperature and the second temperature can be less than 25 degrees Celsius, less than 20 degrees Celsius, less than 15 degrees Celsius, less than 10 degrees Celsius, less than 5 degrees Celsius, or less than 2 degrees Celsius. In a more particular embodiment, the temperature difference between the first temperature and the second temperature can be less than 1 degree Celsius. In yet a more particular embodiment, the temperature difference between the first temperature and the second temperature can be approximately 0 degrees Celsius.

In an embodiment, the operational fluid is a drilling fluid. The drilling fluid can be received from an annulus of a wellbore during drilling operations. In an embodiment, the operational fluid can be production fluids produced from the wellbore. In another embodiment, the operational fluid can be a carrier fluid that is received from the wellbore during a testing, completion, recompletion, decommissioning, other step, or combination thereof.

The extracted gas can include vapor remaining in vapor form as the extracted gas passes through the flow passage to the gas analyzer. In an embodiment, the gas analyzer comprises a sensor that is adapted to analyze the sample of extracted gas while the extracted gas contains vapor. The sensor can be adapted to transmit sensor data to a controller for further analysis. The sensor data can correspond to an analyzed data of the extracted gas.

In certain instances, the sensor and the gas analyzer can be positioned in the flow of the operational fluid within an annulus of the wellbore. In another instance, the sensor and the gas analyzer can be positioned in the flow of the operational fluid downstream from the annulus.

In an embodiment, the extracted gas is not subjected to conditioning between the gas extractor and the gas analyzer. In a more particular embodiment, the extracted gas is not subjected to heating as the extracted gas passes through the flow passage from the gas extractor to the gas analyzer.

In another more particular embodiment, the extracted gas can travel less than 20 feet from the gas extractor to the gas analyzer. That is, for instance, the gas analyzer can be disposed within 20 feet of the gas extractor, within 15 feet of the gas extractor, within 10 feet of the gas extractor, within 5 feet of the gas extractor, or within 3 feet of the gas extractor.

In an embodiment, the gas analyzer can include one sensor. In another embodiment, the gas analyzer can include a plurality of sensors, such as at least two sensors, at least three sensors, at least four sensors, or at least five sensors. In an embodiment, the gas analyzer can be adapted to analyze the extracted gas using at least one of an infrared sensor, a laser sensor, an ultraviolet sensor, a light sensor, a mass spectrometer, a radio frequency detector, an acoustic sensor, an infrared spectrometer, a photoionization detector, an electrochemical gas sensor, an ultrasonic sensor, a photoionization detector, a combustible gas sensor, a semiconductor sensor, a catalytic bead sensor, raman spectroscopy, a Fourier-transform infrared spectroscopy (FTIR), and a flame ionization detector.

In an embodiment, the gas analyzer can include a plurality of analyzing chambers, such as at least two analyzing chambers, at least three analyzing chambers, at least four analyzing chambers, or at least five analyzing chambers. In another embodiment, the gas analyzer can include a single analyzing chamber.

In certain instances, the gas analyzer is in electronic communication with a logic device adapted to process information received from the gas analyzer and use the processed information to perform an operation. The operation can include at least one of relaying the processed information to an operator and affecting an operation of the drilling assembly in response to the processed information.

In an embodiment, the extracted gas can be drawn into the gas analyzer by positive pressure. In another embodiment, the extracted gas can be drawn into the gas analyzer by negative pressure. In yet another embodiment, the extracted gas can be drawn into the gas analyzer by both negative pressure and positive pressure.

In an embodiment, the extracted gas can exit the gas analyzer and be vented to the atmosphere, returned to the drilling fluid, conveyed to a device for storage, moved to another device for further analysis, or any combination thereof.

In certain instances, the system can be at least partially automated. That is, the system can include at least one automated operational step. In another embodiment, the system can be fully automated.

In accordance with another aspect, a method for conducting a subterranean operation can include receiving an operational fluid from a wellbore, with the operational fluid containing a gas and vapor. The method can further include receiving the operational fluid at an input of a gas extractor. The gas can be extracted from the operational fluid with the extracted gas containing the vapor. The extracted gas can be transported through a flow passage to a gas analyzer with the vapor remaining in vapor form as the extracted gas flows through the flow passage. The extracted gas, or a sample thereof, can be analyzed containing the vapor.

In an embodiment, the temperature of the extracted gas is maintained such that the vapor remains in vapor form through the flow passage and into the sample chamber of a gas analyzer where a sensor can analyze the extracted gas.

Receiving the operational fluid at the gas extractor can further include receiving the operational fluid from a mud-gas separator. The mud-gas separator can include an operational fluid conditioning system (e.g. a shaker, a possum bellow, etc.), a flow line, a suction pit, another known mud-gas separator, or any combination thereof.

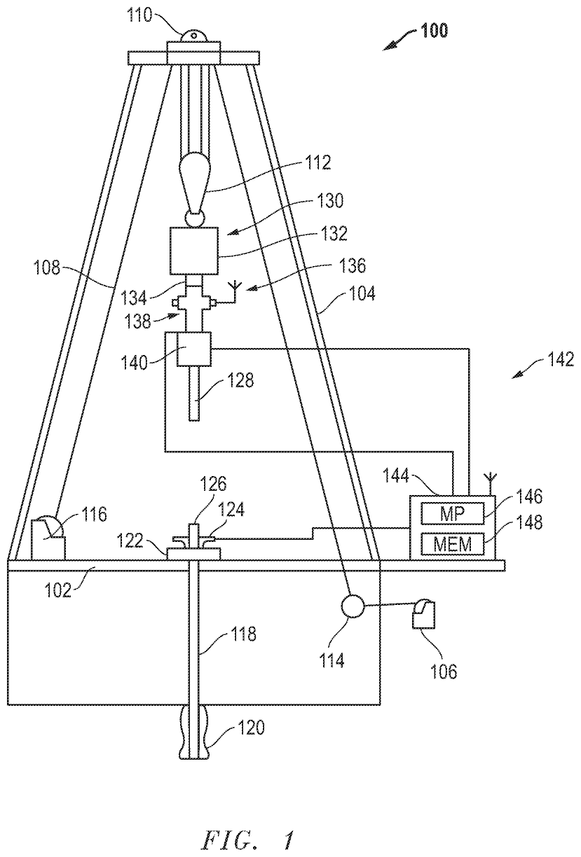

FIG. 1 illustrates an exemplary schematic view of a drilling rig 100 in the process of drilling a wellbore in accordance with an embodiment. The drilling rig 100 can include a drill rig floor 102 and a mast or derrick 104 extending above the rig floor 102. A supply reel 106 can supply drilling line 108 to a crown block 110 and traveling block 112 configured to hoist various types of drilling equipment above the rig floor 102. The drilling line 108 can be secured to a deadline anchor 114. A drawworks 116 can regulate the amount of drilling line 108 in use and, consequently, the height of the traveling block 112 at a given moment. Below the rig floor 102, a drill string 118 can extend downward into a wellbore 120. The drill string 118 can be held stationary with respect to the rig floor 102, for example, by a rotary table 122 and slips 124 (e.g., power slips). A portion of the drill string 118 can extend above the rig floor 102, forming a stump 126 to which another length of tubular 128 (e.g., a joint of drill pipe or a section of casing) may be added.

In an embodiment, a tubular drive system 130, hoisted by the travelling block 112, can position the tubular 128 above the wellbore 120. The tubular drive system 130 can include a top drive 132, a quill 134 (e.g., a sub, a gripping device), and a torque turn system 136 (e.g., a wireless torque turn system, a tubular monitoring system) configured to monitor, control, or evaluate forces acting on the tubular drive system 130, such as torque, weight, and so forth. The quill 134 can extend from the top drive 132 toward the rig floor 102. The torque turn system 136 can measure forces acting on the tubular drive system 130 via a span block 138 (e.g., a sub). In an embodiment, the span block 138 can include a sensor, such as strain gauges, gyroscopes, pressure sensors, accelerometers, magnetic sensors, optical sensors, or other sensors, which may be communicatively linked or physically integrated with the torque turn system 136. Moreover, in certain embodiments, the span block 138 (e.g., via the torque turn system 136) can be coupled to the top drive 132 at a first end (e.g., via the quill 134) and to a casing drive system 140 (e.g., a tubular handling system) at a second end. In certain embodiments, the torque turn system 136 may not be utilized and the span block 138 may be directly coupled between the quill 134 and the casing drive system 140 or tubular 128. The tubular drive system 130, once coupled with the tubular 128, can lower the coupled tubular 128 toward the stump 126 and rotate the tubular 128 such that it connects with the stump 126 and becomes part of the drill string 118.

The drilling rig 100 can further include a control system 142 configured to control various systems and components of the drilling rig 100 that grip, lift, release, and support the tubular 128 and the drill string 118 during a casing running or tripping operation. For example, the control system 142 can control operation of the casing drive system 140 and the slips 124 based on measured feedback (e.g., from the torque turn system, from the span block, from other sensors) to ensure that the tubular 128 and drill string 118 are adequately gripped and supported by the casing drive system 140, the torque turn system 136, the tubular drive system 130, or the slips 124 during a casing running operation. In this manner, the control system 142 can reduce or eliminate incidents where lengths of the tubular 128 or drill string 118 are unsupported. Moreover, the control system 142 can control auxiliary equipment such as mud pumps, robotic pipe handlers, and the like.

In the illustrated embodiment, the control system 142 includes a controller 144 having one or more microprocessors 146 and a memory 148. For example, the controller 144 can be an automation controller which may include a programmable logic controller (PLC). The memory 148 can be a non-transitory (not merely a signal), tangible, computer-readable media, which may include executable instructions that may be executed by the microprocessor 56. The controller 144 can receive feedback from the torque turn system 136 or other sensors that detect measured feedback associated with operation of the drilling rig 100. For example, the controller 144 may receive feedback from the tubular drive system 130 or other sensors in wired or wireless transmission. Based on the measured feedback, the controller 144 can regulate operation of the tubular drive system 130 (e.g., rotation speed, weight on bit).

During operation, the traveling block 112 can be configured to move up and down relative to the rig floor 102. For example, the traveling block 112 can move up to remove the tubular 128 from the drill string 118 or move down to add the tubular 128 to the drill string 118.

While the above described drilling rig 100 may be used with the system described herein, skilled artisans will recognize after reading the entire disclosure that other drilling systems can be used with the systems described herein. For instance, in another embodiment, the drilling rig can include a rack and pinion style drive mechanism, a kelly and rotary table, or another known drilling system.

During certain operations, such as when drilling into the wellbore 120, the subterranean formation can release materials into the wellbore 120. These materials can include, for instance, clippings from the formation and gases and liquids trapped in the formation. After releasing from the formation, these materials can mix with operational fluid from the drilling rig 100 and circulate up the wellbore 120 (e.g., within an annulus defined between the drill string 118 and the wellbore 120) to the surface. The operational fluid containing the circulated materials can include a drilling fluid. The drilling fluid can be received from the annulus of the wellbore 120 during drilling operations. The drilling fluid can be biased to the surface, for example, by one or more mud pumps. Drilling fluid can be circulated down the drill string 118 and return through the annulus. The operational fluid can further include production fluid that is produced from the wellbore 120. The operational fluid can further include a carrier fluid that is received from the wellbore 120 during a completion operation, such as a gravel packing operation.

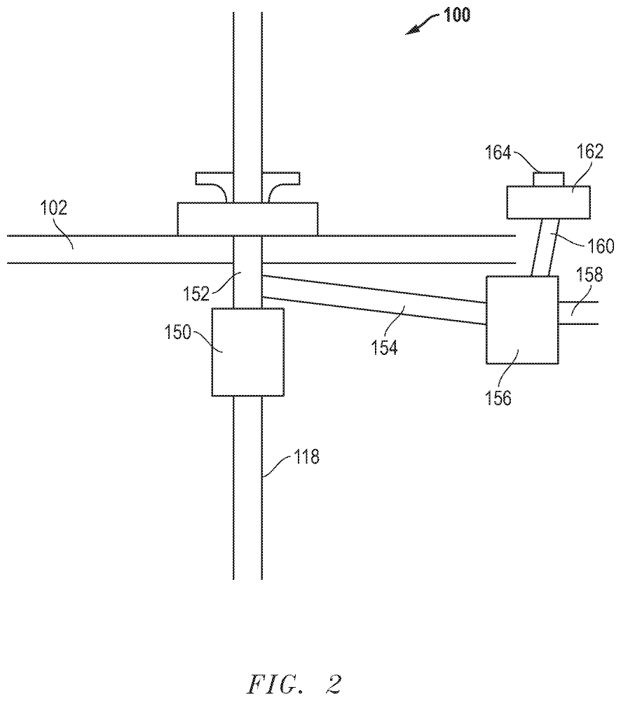

FIG. 2 includes an enlarged schematic view of a portion of the drilling rig 100. In the illustrated embodiment, the drill string 118 is in communication with a blowout preventer (BOP) 150. The BOP 150 can include an annular type BOP, a pipe ram and blind ram type BOP, or both. A bell nipple 152 can be disposed between the BOP 150 and the rig floor 102. The bell nipple 152 can include a section of large diameter pipe. The bell nipple 152 can include an opening in fluid communication with a flow line 154 extending to a gas extractor 156. The gas extractor 156 can be configured to extract a gas from the operational fluid returning to the surface of the formation. In an embodiment, the gas extractor 156 can be adapted to extract a sample of gas from the operational fluid. The sample can be in a range of 0.01% and 100% of the volumetric density of gas within the operational fluid. In certain instances, sampling can be adjusted based on system requirements discussed hereinafter.

A mud line 158 can extend from the gas extractor 156 to a mud pit or other mud storage area (not illustrated) for reuse circulating operational fluid within the wellbore 120. In an embodiment, operational fluid transported through the mud line 158 can have a lower gas concentration as compared to the operational fluid passing through the flow line 154. For instance, the operational fluid can have a first volumetric density of gas within the flow line 154 and a second volumetric density of gas within the mud line 158, where the second volumetric density is less than the first volumetric density. In a more particular embodiment, operational fluid transported through the mud line 158 can be free, or essentially free, of gas extracted by the gas extractor 156.

In the illustrated embodiment, the gas extractor 156 is adapted to receive operational fluid from the flow line 154. In an embodiment, the gas extractor 156 can be adapted to extract gas from the operational fluid prior to the operational fluid entering an operational fluid conditioning system (e.g. a shale shaker, a possum belly, distribution box, a suction pit, sand traps, shaker boxes, header boxes, or from a flowline trap disposed at the head of the shale shaker). In another embodiment, the gas extractor 156 can be adapted to extract gas from the operational fluid after the operational fluid passes through a mud-gas separator, such as a shale shaker. In yet a further embodiment, the gas extractor 156 can be adapted to extract gas from the operational fluid after passing through a drilling fluids header box (not illustrated).

Gas extracted from the operational fluid by the gas extractor 156 can be transported through a flow passage 160 to a gas analyzer 162. In the illustrated embodiment, the gas analyzer 162 is disposed above the rig floor 102. In another embodiment, the gas analyzer 162 can be disposed within the rig floor 102, below the rig floor 102, or to a lateral side of the rig floor 102. In certain embodiments, the gas analyzer 162 is disposed within 20 feet of the gas extractor 156, within 15 feet of the gas extractor 156, within 10 feet of the gas extractor 156, within 5 feet of the gas extractor 156, or within 3 feet of the gas extractor 156. In a particular embodiment, the gas analyzer 156 can be disposed within 2 feet of the gas extractor 156, or within 1 foot of the gas extractor 156. In another embodiment, the gas analyzer 156 can be integral with the gas extractor 156.

In an embodiment, extracted gas passing through the flow passage 160 may be unconditioned. That is, extracted gas within the flow passage 160 may not be subjected to conditioning, such as heating, cooling, drying, or any combination thereof. In this manner, the unconditioned, extracted gas can be received at the gas analyzer 162 without significant alteration of the properties thereto. In another embodiment, the extracted gas may be conditioned to maintain certain properties thereof. For instance, the flow line 154, gas extractor 156, flow passage 160, or gas analyzer 162 can include a heating element to maintain a temperature of the extracted gas within a suitable range. In both conditioned and unconditioned applications, extracted gas can pass through one or more filtrations devices, such as one or more screens, filters, or webbings adapted to remove particulates and other solids from the extracted gas.

The gas extractor 156 can be configured to extract gas from the operational fluid at a first temperature. The extracted gas can contain vapor when it exits the gas extractor 156 and enters the flow passage 160. The gas analyzer 162 can receive the extracted gas from the flow passage at a second temperature. In an embodiment, a temperature difference between the first and second temperatures can be less than 30 degrees Celsius. In a more particular embodiment, the temperature difference between the first temperature and the second temperature can be less than 25 degrees Celsius, less than 20 degrees Celsius, less than 15 degrees Celsius, less than 10 degrees Celsius, less than 5 degrees Celsius, or less than 2 degrees Celsius. In yet a more particular embodiment, the temperature difference between the first temperature and the second temperature can be less than 1 degree Celsius. In yet another more particular embodiment, the temperature difference between the first temperature and the second temperature can be approximately 0 degrees Celsius. Maintenance of a low temperature difference between the first temperature and the second temperature can prevent precipitation of the vapor within the extracted gas. In a particular instance, sensing of the extracted gas by the gas analyzer 162 can be performed without temperature compensation.

Vapor can remain in vapor form in the extracted gas as the extracted gas passes through the flow passage 160 to the gas analyzer 162. For instance, the extracted gas can have a first vapor content, WV₁, when it is extracted from the operational fluid and a second vapor content, WV₂, when it is received at the gas analyzer 162. In an embodiment, WV₂ can be no less than 0.5 WV₁, no less than 0.55 WV₁, no less than 0.6 WV₁, no less than 0.65 WV₁, no less than 0.7 WV₁, no less than 0.75 WV₁, no less than 0.8 WV₁, or no less than 0.85 WV₁. In a more particular embodiment, WV₂ can be no less than 0.9 WV₁, no less than 0.95 WV₁, no less than 0.96 WV₁, no less than 0.97 WV₁, no less than 0.98 WV₁, or no less than 0.99 WV₁. In a more particular embodiment, WV₂ can be equal, or approximately equal, to WV₁.

Extracted gas can be drawn into the gas analyzer 162 from the flow passage 160 by positive pressure, negative pressure, or both. In an embodiment, the gas analyzer 162 can include a sensor 164 adapted to analyze the extracted gas containing vapor. In a particular instance, the sensor 164 can be adapted to analyze only a sample of extracted gas passing through the gas analyzer 162. In another instance, the sensor 164 can analyze the entire volume of extracted gas passing through the gas analyzer 162. By way of non-limiting example, the sensor 164 can include an infrared sensor, a laser sensor, an ultraviolet sensor, a light sensor, a mass spectrometer, a radio frequency detector, an acoustic sensor, an infrared spectrometer, a photoionization detector, an electrochemical gas sensor, an ultrasonic sensor, a photoionization detector, a combustible gas sensor, a semiconductor sensor, a catalytic bead sensor, raman spectroscopy, a Fourier-transform infrared spectroscopy (FTIR), a flame ionization detector, or any combination thereof.

In certain instances, the sensor 164 can include a multipart construction. For instance, the sensor 164 can include an interfacing portion adapted to be disposed near the extracted gas (e.g., near or within a volume adapted to contain the extracted gas) and a sensing portion adapted to sense the extracted gas. In an embodiment, the interfacing portion and sensing portion of the sensor 164 can be part of a same general structure. In another embodiment, the interfacing portion and sensing portion can be part of different portions. For instance, the interfacing portion and sensing portion can be spaced apart from one another. By way of non-limiting example, the interfacing portion can include a laser coupled with the sensing portion by a cable, such as a fiber optic cable. The laser can transport the gas sample, or a signature thereof, through the cable to the remotely located sensing portion.

In certain embodiments, the gas analyzer 162 can include a single analyzing chamber adapted to analyze the extracted gas. The sensor 164 can be disposed in fluid communication with the single analyzing chamber and can be adapted to sense one or more characteristics or properties of the extracted gas. In another embodiment, the gas analyzer 162 can include a plurality of analyzing chambers. One or more sensors 164 can be in fluid communication with at least one of the plurality of analyzing chambers and adapted to sense the one or more characteristics or properties of the extracted gas.

In an embodiment, the gas analyzer 162 can be in electronic communication with a controller, such as the controller 144, another controller, or a combination thereof. The sensor 164 can be adapted to transmit sensor data to the controller for further analysis. The gas analyzer 162 can be in electronic communication with a logic device, such as a logic device of the controller, adapted to process information received from the gas analyzer 162. The logic device can use the processed information to perform an operation. In an embodiment, the operation can include relaying the processed information to an operator. In another embodiment, the operation can include affecting an operation of the drilling assembly in response to the processed information.

Extracted gas can exit the gas analyzer 162 via a vent. The gas can be vented, for example, to the atmosphere, returned to the drilling fluid, conveyed to a device for storage, moved to another device for further analysis, or any combination thereof.

In certain instances, one or more of the systems, components, or tools described herein can be automated. For instance, the control of the flow line 154, mud line 158, or flow passage 160 can be performed by one or more valves or other flow restricting elements to permit passage of an effective volume of gas to the gas analyzer 162. The one or more valves or other flow restricting elements can be automated to provide an effective volume of gas to the gas analyzer 162.

Use of a gas analyzer 162 adapted to sense conditions of the extracted gas with vapor remaining therein can reduce erroneous gas profile readings caused by gas conditioning and other modifying operations. Specifically, gas conditioning can alter or change certain characteristics of the gas, including the actual gas concentrations. Reduction in conditioning can permit higher accuracy gas analysis. In certain instances, the gas analyzer 162 can be adapted to analyze the extracted gas from the wellbore in a shorter time period as compared to analysis requiring conditioning. This can enhance predictive operations and reduce time delay required for gas conditioning, allowing a drilling operator or microprocessor to more quickly assess drilling and wellbore factors to optimize or alter the well plan or trajectory of the drill string.

It should be noted that the illustrations are intentionally simplified. Many other components and tools may be employed during the various periods of formation and preparation of the wellbore. Moreover, some components and tools may be omitted during various periods of formation and preparation of the wellbore. Similarly, as will be appreciated by those skilled in the art, the orientation and environment of the well may vary widely depending upon the location and situation of the formations of interest. For example, rather than a generally vertical bore, the wellbore, in practice, may include one or more deviations, including angled and horizontal runs. Similarly, while shown as a surface (land-based) operation, the wellbore may be formed in water of various depths, in which case the topside equipment may include an anchored or floating platform. While only certain features of the invention have been illustrated and described herein, many modifications and changes will occur to those skilled in the art. It is, therefore, to be understood that the claims are intended to cover all such modifications and changes as fall within the true spirit of the invention.

### Embodiment 1

A system for use in subterranean operations, the system comprising:
a gas extractor configured to extract a gas at a first temperature from an operational fluid, with the extracted gas containing vapor when it exits the gas extractor into a flow passage; and
a gas analyzer that receives the gas at a second temperature from the flow passage, wherein the temperature difference between the first temperature and the second temperature is less than 30 degrees Celsius.

### Embodiment 2

The system of embodiment 1, wherein the operational fluid is a drilling fluid.

### Embodiment 3

The system of embodiment 2, wherein the drilling fluid is received from an annulus of a wellbore during a drilling operation.

### Embodiment 4

The system of embodiment 1, wherein the operational fluid is production fluid that is produced from a wellbore.

### Embodiment 5

The system of embodiment 1, wherein the operational fluid is a carrier fluid that is received from a wellbore during a gravel pack operation.

### Embodiment 6

The system of any one of the preceding embodiments, wherein the vapor remains in vapor form in the extracted gas as the extracted gas passes through the flow passage to the gas analyzer.

### Embodiment 7

The system of embodiment 6, wherein the gas analyzer comprises a sensor that analyzes a sample of the extracted gas containing the vapor.

### Embodiment 8

The system of embodiment 7, wherein the sensor transmits sensor data to a controller for further analysis.

### Embodiment 9

The system of embodiment 7, wherein the sensor and the gas analyzer are positioned in the flow of the operational fluid within an annulus or downstream from the annulus.

### Embodiment 10

The system of any one of the preceding embodiments, wherein the extracted gas is not subjected to heating as the extracted gas passes through the flow passage to the gas analyzer.

### Embodiment 11

The system of any one of the preceding embodiments, wherein the gas analyzer is disposed within 20 feet of the gas extractor, within 15 feet of the gas extractor, within 10 feet of the gas extractor, within 5 feet of the gas extractor, or within 3 feet of the gas extractor.

### Embodiment 12

The system of any one of the preceding embodiments, wherein the temperature difference between the first temperature and the second temperature is less than 25 degrees Celsius, less than 20 degrees Celsius, less than 15 degrees Celsius, less than 10 degrees Celsius, less than 5 degrees Celsius, or less than 2 degrees Celsius.

### Embodiment 13

The system of any one of the preceding embodiments, wherein the temperature difference between the first temperature and the second temperature is less than 1 degree Celsius.

### Embodiment 14

The system of any one of the preceding embodiments, wherein the temperature difference between the first temperature and the second temperature is approximately 0 degrees Celsius.

### Embodiment 15

The system of any one of the preceding embodiments, wherein the gas analyzer is adapted to analyze the extracted gas using at least one of an infrared sensor, a laser sensor, an ultraviolet sensor, a light sensor, a mass spectrometer, a radio frequency detector, an acoustic sensor, an infrared spectrometer, a photoionization detector, an electrochemical gas sensor, an ultrasonic sensor, a photoionization detector, a combustible gas sensor, a semiconductor sensor, a catalytic bead sensor, raman spectroscopy, a Fourier-transform infrared spectroscopy (FTIR), a flame ionization detector, and hot wire sensor.

### Embodiment 16

The system of any one of the preceding embodiments, wherein the gas analyzer comprises a plurality of analyzing chambers.

### Embodiment 17

The system of any one of the preceding embodiments, wherein the gas analyzer is in electronic communication with a logic device adapted to process information received from the gas analyzer and use the processed information to perform an operation.

### Embodiment 18

The system of embodiment 17, wherein the operation comprises at least one of relaying the processed information to an operator and affecting an operation of the drilling assembly in response to the processed information.

### Embodiment 19

The system of any one of the preceding embodiments, wherein the extracted gas is drawn into the gas analyzer by positive pressure, negative pressure, or both.

### Embodiment 20

The system of any one of the preceding embodiments, wherein extracted gas exiting the gas analyzer is vented to the atmosphere, returned to the drilling fluid, conveyed to a device for storage, moved to another device for further analysis, or any combination thereof.

### Embodiment 21

The system of any one of the preceding embodiments, wherein the system is automated.

### Embodiment 22

The system of any one of the preceding embodiments, wherein the vapor comprises a water vapor, an oil vapor, another vapor, or any combination thereof.

### Embodiment 23

A system for use in subterranean operations, comprising:
a gas extractor configured to extract a gas from an operational fluid; and
a gas analyzer adapted to receive the gas from a passage in communication with the gas extractor and analyze the gas,
wherein the gas is not subjected to conditioning between the gas extractor and gas analyzer.

### Embodiment 24

A method for conducting a subterranean operation, the method comprising:
receiving an operational fluid from a wellbore, with the operational fluid containing a gas and vapor;
receiving the operational fluid at an input of a gas extractor;
extracting the gas from the operational fluid, with the extracted gas containing the vapor;
transporting the extracted gas through a flow passage to a gas analyzer, with the vapor remaining in vapor form as the extracted gas flows through the flow passage; and
analyzing a sample of the extracted gas containing the vapor.

### Embodiment 25

The method of embodiment 24, further comprising maintaining a temperature of the extracted gas such that the vapor remains in vapor form through the flow passage and into a sample chamber where a sensor analyzes the extracted gas.

### Embodiment 26

The method of embodiment 25, wherein the receiving the operational fluid at the gas extractor further comprises receiving the operational fluid from a mud-gas separator.

### Embodiment 27

The method of embodiment 25, wherein the receiving the operation fluid at the gas extractor further comprises receiving the operational fluid from a flow line prior to the operational fluid entering a possum belly.

### Embodiment 28

The method of embodiment 25, wherein the receiving the operation fluid at the gas extractor further comprises receiving the operational fluid from a drilling fluids header box.

Note that not all of the activities described above in the general description or the examples are required, that a portion of a specific activity may not be required, and that one or more further activities may be performed in addition to those described. Still further, the order in which activities are listed is not necessarily the order in which they are performed.

Benefits, other advantages, and solutions to problems have been described above with regard to specific embodiments. However, the benefits, advantages, solutions to problems, and any feature(s) that may cause any benefit, advantage, or solution to occur or become more pronounced are not to be construed as a critical, required, or essential feature of any or all the claims.

The specification and illustrations of the embodiments described herein are intended to provide a general understanding of the structure of the various embodiments. The specification and illustrations are not intended to serve as an exhaustive and comprehensive description of all of the elements and features of apparatus and systems that use the structures or methods described herein. Separate embodiments may also be provided in combination in a single embodiment, and conversely, various features that are, for brevity, described in the context of a single embodiment, may also be provided separately or in any subcombination. Further, reference to values stated in ranges includes each and every value within that range. Many other embodiments may be apparent to skilled artisans only after reading this specification. Other embodiments may be used and derived from the disclosure, such that a structural substitution, logical substitution, or another change may be made without departing from the scope of the disclosure. Accordingly, the disclosure is to be regarded as illustrative rather than restrictive.

1. A system for use in subterranean operations, the system comprising:
a gas extractor configured to extract a gas at a first temperature from an operational fluid, with the gas containing a vapor when it exits the gas extractor into a flow passage; and
a gas analyzer that receives the gas at a second temperature from the flow passage, wherein a temperature difference between the first temperature and the second temperature is less than 30 degrees Celsius.

2. The system of claim 1, wherein the operational fluid is a drilling fluid, or wherein the operational fluid is production fluid that is produced from a wellbore, or wherein the operational fluid is a carrier fluid that is received from a wellbore during a gravel pack operation.

3. The system of claim 2, wherein the drilling fluid is received from an annulus of a wellbore during a drilling operation.

4. The system of claim 1, wherein the vapor remains in vapor form in the extracted gas as the extracted gas passes through the flow passage to the gas analyzer.

5. The system of claim 4, wherein the gas analyzer comprises a sensor that analyzes a sample of the extracted gas containing the vapor, and wherein the sensor transmits sensor data to a controller for further analysis.

6. The system of claim 5, wherein the sensor and the gas analyzer are positioned in flow of the operational fluid within an annulus or downstream from the annulus.

7. The system of claim 1, wherein the extracted gas is not subjected to heating as the extracted gas passes through the flow passage to the gas analyzer.

8. The system of claim 1, wherein the gas analyzer is disposed within 20 feet of the gas extractor, within 15 feet of the gas extractor, within 10 feet of the gas extractor, within 5 feet of the gas extractor, or within 3 feet of the gas extractor.

9. The system of claim 1, wherein the temperature difference between the first temperature and the second temperature is less than 25 degrees Celsius, less than 20 degrees Celsius, less than 15 degrees Celsius, less than 10 degrees Celsius, less than 5 degrees Celsius, or less than 2 degrees Celsius.

10. The system of claim 1, wherein the temperature difference between the first temperature and the second temperature is less than 1 degree Celsius.

11. The system of claim 1, wherein the temperature difference between the first temperature and the second temperature is approximately 0 degrees Celsius.

12. The system of claim 1, wherein the gas analyzer is adapted to analyze the extracted gas using at least one of an infrared sensor, a laser sensor, an ultraviolet sensor, a light sensor, a mass spectrometer, a radio frequency detector, an acoustic sensor, an infrared spectrometer, a photoionization detector, an electrochemical gas sensor, an ultrasonic sensor, a photoionization detector, a combustible gas sensor, a semiconductor sensor, a catalytic bead sensor, raman spectroscopy, a Fourier-transform infrared spectroscopy (FTIR), a flame ionization detector, and hot wire sensor.

13. The system of claim 1, wherein the gas analyzer is in electronic communication with a logic device adapted to process information received from the gas analyzer and use the processed information to perform an operation, and wherein the operation comprises at least one of relaying the processed information to an operator and affecting an operation of a drilling assembly in response to the processed information.

14. The system of claim 1, wherein extracted gas exiting the gas analyzer is vented to an external atmosphere, returned to the operational fluid, conveyed to a device for storage, moved to another device for further analysis, or any combination thereof.

15. A system for use in subterranean operations, comprising:
a gas extractor configured to extract a gas from an operational fluid; and
a gas analyzer adapted to receive the gas from a passage in communication with the gas extractor and analyze the gas,
wherein the gas is not subjected to conditioning between the gas extractor and gas analyzer.

16. The system of claim 15, wherein the operational fluid is a drilling fluid, or wherein the operational fluid is production fluid that is produced from a wellbore, or wherein the operational fluid is a carrier fluid that is received from a wellbore during a gravel pack operation.

17. A method for conducting a subterranean operation, the method comprising:
receiving an operational fluid from a wellbore, with the operational fluid containing a gas and vapor;
receiving the operational fluid at an input of a gas extractor;
extracting the gas from the operational fluid, with the extracted gas containing the vapor;
transporting the extracted gas through a flow passage to a gas analyzer, with the vapor remaining in vapor form as the extracted gas flows through the flow passage; and
analyzing a sample of the extracted gas containing the vapor.

18. The method of claim 17, further comprising maintaining a temperature of the extracted gas such that the vapor remains in vapor form through the flow passage and into a sample chamber where a sensor analyzes the extracted gas.

19. The method of claim 18, wherein the receiving the operational fluid at the gas extractor further comprises receiving the operational fluid from a mud-gas separator.

20. The method of claim 18, wherein the receiving the operation fluid at the gas extractor further comprises receiving the operational fluid from a flow line prior to the operational fluid entering an operational fluid conditioning system.
